(19) United States
(12) Patent Application Publication
(10) Pub. No.: US 2020/0151634 A1
(43) Pub. Date: May 14, 2020

(54) Methods for Probabilistic Demand/Supply Matching and Designing Scheduling Decision Support Systems and Schedulers

(71) Applicant: EasyManage Health, Blaine, MN (US)

(72) Inventors: Fitih Cinnor, Plymouth, MN (US); Nayeem Ahmed, Blaine, MN (US); Sandeep Gupta, North Oaks, MN (US)

(73) Assignee: EasyManage Health, Blaine, MN (US)

(21) Appl. No.: 16/679,250

(22) Filed: Nov. 10, 2019

Related U.S. Application Data
(60) Provisional application No. 62/758,553, filed on Nov. 10, 2018.

(51) Int. Cl. G06Q 10/06; G06Q 30/02; G16H 40/20; G06N 20/00

(52) U.S. Cl. CPC G06Q 10/06312; G06Q 10/0637; G06N 20/00; G16H 40/20; G06Q 30/0204; G06Q 30/0201

(57) ABSTRACT

Apparatus and associated methods relate to scheduling service provider and service consumer interactions based on determining service consumer scores, service provider scores, and service time slot scores predicting outcomes for provider service to a consumer in a time slot, and automatically adapting demand and supply matched as functions of the scores. In an illustrative example, the service consumer may be a client. The service provider may be, for example, a professional offering service in an available time slot. In some examples, individual client, provider, and time slot scores may be calculated as functions of predictive variables associated with a client population and the provider practice environment. In some embodiments, scores may be probability estimates of show, no-show, delay, or cancellation. Various embodiments may advantageously determine schedules with maximum likelihood of full occupancy to optimize resource utilization and revenue expenditure, based on collectively optimizing client, provider, and time slot probability estimates.

### CROSS-REFERENCE TO RELATED APPLICATIONS

This application claims the benefit of U.S. Provisional Application No. 62/758,553, titled “Methods for Probabilistic Demand/Supply Matching and Designing Scheduling Decision Support Systems and Schedulers,” Inventors: Fitih M. Cinnor, Nayeem Ahmed, and Sandeep Gupta; filed by: Applicants Fitih M. Cinnor, Nayeem Ahmed, and Sandeep Gupta, on 11 Oct. 2018.

This application incorporates the entire contents of the above-referenced application herein by reference.

### TECHNICAL FIELD

Various embodiments relate generally to demand/supply matching.

### BACKGROUND

Demand is the quantity of an item demanded by consumers. Supply is the quantity of the demanded item supplied by providers. In some examples, the item demanded may be a physical article. For example, an automobile may be the subject of consumer demand. In an illustrative example, some articles subject to consumer demand may be services, such as, for example, transportation. Some demand scenario examples include a service offered by a service provider to consumers. In some exemplary scenarios, matching a service provider to a service consumer may result in a successful service outcome.

Some service providers may be professional service providers. For example, medical doctors and attorneys may offer professional services, including medical or legal services, to consumers. In an illustrative example, consumers of medical services may include medical patients. In various scenarios, a medical doctor may offer medical services to patients as part of a medical practice. Some exemplary medical practice scenarios may include multiple medical doctors offering a variety of medical services to a patient population at diverse locations and times. In various examples, a medical practice offering a variety of medical services to a patient population may expend significant effort matching supply and demand.

### SUMMARY

Apparatus and associated methods relate to scheduling service provider and service consumer interactions based on determining service consumer scores, service provider scores, and service time slot scores predicting outcomes for provider service to a consumer in a time slot, and automatically adapting demand and supply matched as functions of the scores. In an illustrative example, the service consumer may be a client. The service provider may be, for example, a professional offering service in an available time slot. In some examples, individual client, provider, and time slot scores may be calculated as functions of predictive variables associated with a client population and the provider practice environment. In some embodiments, scores may be probability estimates of show, no-show, delay, or cancellation. Various embodiments may advantageously determine schedules with maximum likelihood of full occupancy to optimize resource utilization and revenue expenditure, based on collectively optimizing client, provider, and time slot probability estimates.

Various embodiments may achieve one or more advantages. For example, some embodiments may improve a user's ease of determining schedules for service provider and service consumer interactions. This facilitation may be a result of reducing the user's effort optimizing resource utilization and revenue expenditure based on collectively optimizing client, provider, and time slot probability estimates. In some embodiments, service delivery efficiency may be automatically increased for a user's practice. Such automatically increased service delivery efficiency may reduce a user's exposure to excess cost and reduced profit. Various designs may increase client satisfaction with a user's practice. Such increased client satisfaction may be a result of an embodiment practice management implementation automatically optimizing consumer demand and provider supply to minimize consumer and provider no-shows, delays, and cancelations. Some embodiments may act to increase demand for a user's available service provider time slots with notifications sent to clients encouraging the client to seek service in an available time slot. Such automatic client notification of available service time slots may increase profit from the user's practice and improve the client's experience. For example, a patient notified that a doctor may be available earlier than anticipated may receive medical attention more quickly, improving the accuracy or usefulness of medical diagnosis or treatment.

In some embodiments, the effort required by a user to efficiently schedule provider resources to serve the user's service consumers may be reduced. For example, in various embodiment practice management implementations, a user's medical practice that employs many medical doctors may optimize their doctor's work schedules based on assigning clients, providers, and time slots according to scores calculated as a function of predictive variables of a patient population and the medical practice. Some embodiments may improve practice resource allocation and utilization, increase revenue, and reduce wait time. This facilitation may be a result of an embodiment service rendering environment scheduling events and client and service provider interactions based on optimized probabilistic demand and supply matching.

The details of various embodiments are set forth in the accompanying drawings and the description below. Other features and advantages will be apparent from the description and drawings, and from the claims.

### BRIEF DESCRIPTION OF THE DRAWINGS

FIG. 1 depicts an illustrative operational process scheduling service provider and service consumer interactions based on determining service consumer scores, service provider scores, and service time slot scores predicting outcomes for provider service to a consumer in a time slot, and automatically adapting demand and supply matched as functions of the scores.

FIG. 2 depicts a schematic view of an exemplary service provider and service consumer network configured to implement a process scheduling service provider and service consumer interactions based on determining service consumer scores, service provider scores, and service time slot scores predicting outcomes for provider service to a consumer in a time slot, and automatically adapting demand and supply matched as functions of the scores.

FIG. 3 depicts a structural view of an exemplary computing device adapted with an embodiment Demand/Supply Matching Engine (DSME) configured to execute a process scheduling service provider and service consumer interactions based on determining service consumer scores, service provider scores, and service time slot scores predicting outcomes for provider service to a consumer in a time slot, and automatically adapting demand and supply matched as functions of the scores.

FIG. 4 depicts an illustrative process flow of an exemplary Demand/Supply Matching Engine (DSME) automatically adapting demand and supply matched as functions of service consumer scores, service provider scores, and service time slot scores, in accordance with some embodiment implementations.

FIG. 5 depicts an illustrative process flow of an exemplary Demand/Supply Matching Engine (DSME) automatically adapting demand and supply matched as functions of service consumer scores, service provider scores, and service time slot scores, in accordance with some embodiment implementations.

FIG. 6 depicts an illustrative process flow of an exemplary Demand/Supply Matching Engine (DSME) automatically adapting demand and supply matched as functions of service consumer scores, service provider scores, and service time slot scores, in accordance with some embodiment implementations.

FIG. 7 depicts an illustrative process flow of an exemplary Demand/Supply Matching Engine (DSME) automatically adapting demand and supply matched as functions of service consumer scores, service provider scores, and service time slot scores, in accordance with some embodiment implementations.

FIG. 8 depicts an illustrative risk heatmap exemplary of an embodiment Demand/Supply Matching Engine (DSME) automatically adapting demand and supply matched as functions of service consumer scores, service provider scores, and service time slot scores, in accordance with some embodiment implementations.

Like reference symbols in the various drawings indicate like elements.

### DETAILED DESCRIPTION OF ILLUSTRATIVE EMBODIMENTS

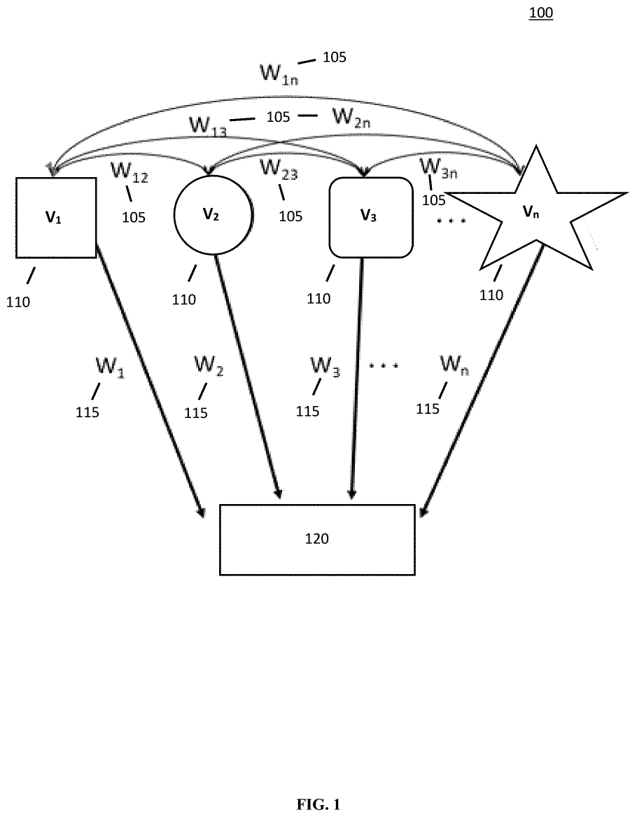

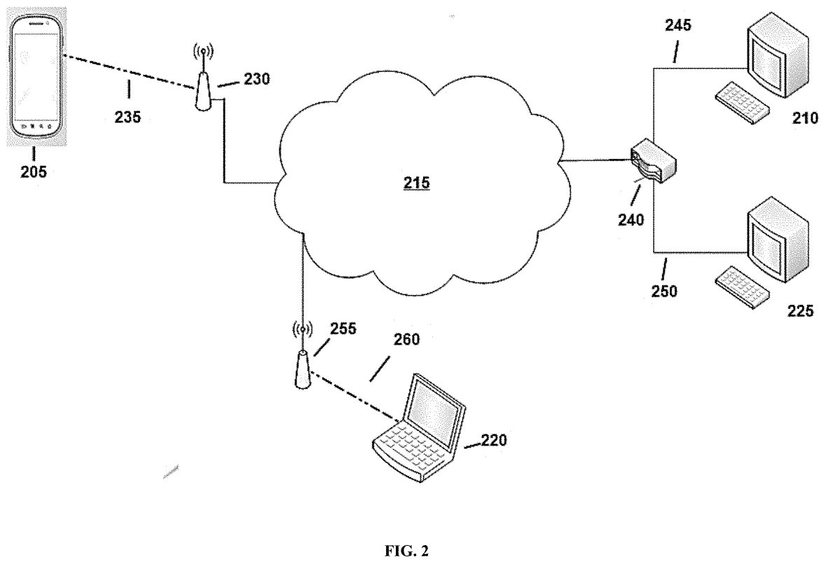

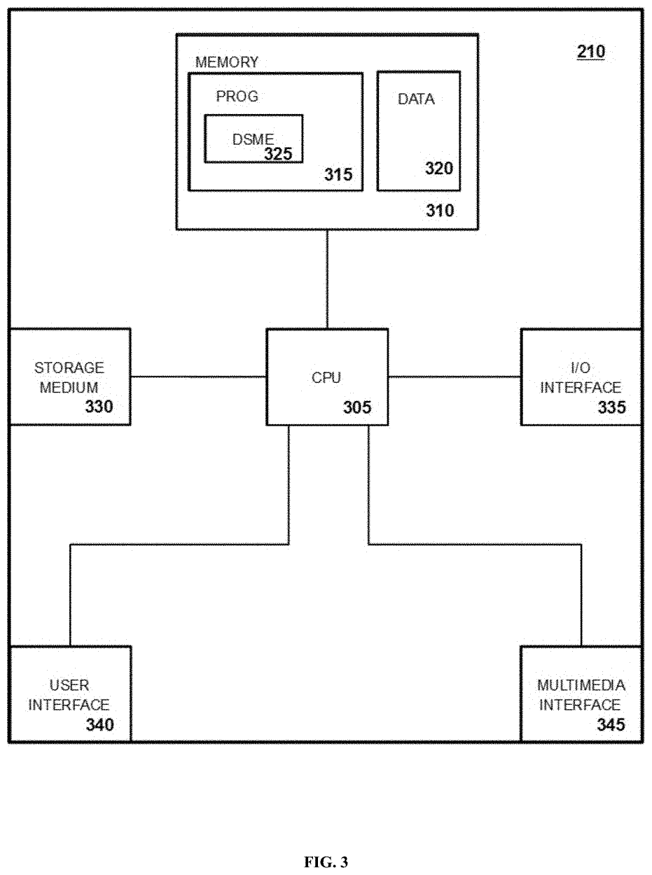

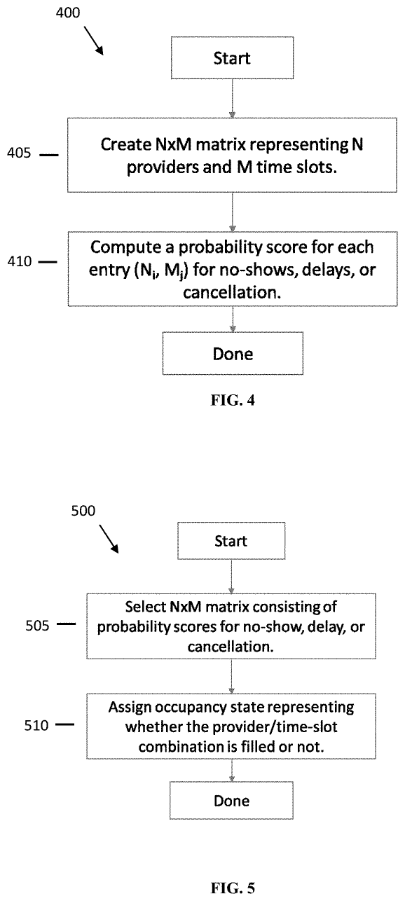

To aid understanding, this document is organized as follows. First, optimized probabilistic demand and supply matching based on automatically adapting demand and supply matched as functions of predictive service consumer scores, service provider scores, and service time slot scores, is briefly introduced with reference to FIG. 1. Second, with reference to FIGS. 2-3, the discussion turns to exemplary embodiments that illustrate optimized probabilistic demand and supply matching implementations. Specifically, illustrative embodiment optimized probabilistic demand and supply matching network and computing device implementations are disclosed. Finally, with reference to FIGS. 4-8, process flow and screenshot views illustrative of an exemplary Demand/Supply Matching Engine (DSME) are described, to explain improvements in demand and supply matching technology.

FIG. 1 depicts an illustrative operational process scheduling service provider and service consumer interactions based on determining service consumer scores, service provider scores, and service time slot scores predicting outcomes for provider service to a consumer in a time slot, and automatically adapting demand and supply matched as functions of the scores. In FIG. 1, the exemplary demand/supply matching process data flow 100 models provider, consumer, and time slot interactions to obtain optimal service outcomes for a user. In some examples, the provider may be a medical doctor. In various scenarios, the consumer may be a medical patient seeking medical attention from the user's medical practice employing the medical doctor. In some examples, one or more provider may offer service during various time slot periods. In the illustrated embodiment, the depicted demand/supply matching process data flow 100 supports probabilistic demand/supply matching determined as a function of an adaptive algorithm optimizing scheduling efficiency based on matching service consumers, service providers, and time slots. In the illustrated example, interactions between consumers, providers, and time slots are modeled to determine a score predicting an outcome characteristic related to each modeled interaction. In an illustrative example, a consumer score may be determined as a function of the consumer's historical no-show probability, the consumer's physical condition, and the consumer's distance from the practice facility where service may be rendered in a given time slot. In some embodiments, a consumer, provider, or time slot score may be determined as a function of a probabilistic estimate of no-shows, delays, or cancellations; practice resource utilization/allocation preferences; demand forecasting; and, other related factors. In the depicted embodiment, the weighting factors 105 determine the contribution of each modeled interaction on the predicted outcome. In the illustrated example, the modeled interactions are characterized by the input variables 110 defining characteristics of the consumers, providers, and time slots. In the depicted embodiment, the weighting factors 115 determine the contribution of each input variable 110 on the output variables 120. In the illustrated example, the output variables 120 represent the score predicting the outcome of an interaction between a consumer, a provider, and a time slot. In an illustrative example, an embodiment medical practice demand/supply matching process may evaluate the predicted consumer, provider, and time slot interaction scores to obtain optimal service outcomes for a user's practice, based on matching patients, doctors, and time slots scheduled as a function of the scores.

FIG. 2 depicts a schematic view of an exemplary service provider and service consumer network configured to implement a process scheduling service provider and service consumer interactions based on determining service consumer scores, service provider scores, and service time slot scores predicting outcomes for provider service to a consumer in a time slot, and automatically adapting demand and supply matched as functions of the scores. In FIG. 2, according to an exemplary embodiment of the present disclosure, data may be transferred to the system, stored by the system and/or transferred by the system to users of the system across local area networks (LANs) or wide area networks (WANs). In accordance with various embodiments, the system may include numerous servers, data mining hardware, computing devices, or any combination thereof, communicatively connected across one or more LANs and/or WANs. One of ordinary skill in the art would appreciate that there are numerous manners in which the system could be configured, and embodiments of the present disclosure are contemplated for use with any configuration. Referring to FIG. 2, a schematic overview of a system in accordance with an embodiment of the present disclosure is shown. In the depicted embodiment, an exemplary system includes the exemplary user device 205 configured to provide a user access to the demand/supply matching system 210 via the network cloud 215. In the depicted example, the user device 205 is a smartphone configured with a mobile app adapted to provide the user access to practice management, demand/supply matching, and scheduling services of the demand/supply matching system 210. In the illustrated embodiment, the practice management system 220 is a computing device configured to manage service providers offering services to service consumers through a professional practice in time slots scheduled for the user's practice by the demand/supply matching system 210. In the illustrated example, the practice management server 225 is a computing device hosting a practice management database and practice management applications to support the demand/supply matching system 210 facilitating practice management, demand/supply matching, and scheduling for the user's practice. In the illustrated embodiment, the user device 205 is communicatively and operably coupled by the wireless access point 230 and the wireless link 235 with the network cloud 215 (for example, the Internet) to send, retrieve, or manipulate information in storage devices, servers, and network components, and exchange information with various other systems and devices via the network cloud 215. In the depicted example, the illustrative system includes the router 240 configured to communicatively and operably couple the demand/supply matching system 210 to the network cloud 215 via the communication link 245. In the illustrated example, the router 240 also communicatively and operably couples the practice management server 225 to the network cloud 215 via the communication link 250. In the depicted embodiment, the practice management system 220 is communicatively and operably coupled with the network cloud 215 by the wireless access point 255 and the wireless communication link 260. In various examples, one or more of: the user device 205, the demand/supply matching system 210, the practice management system 220, or the practice management server 225 may include an application server configured to store or provide access to information used by the system. In various embodiments, one or more application server may retrieve or manipulate information in storage devices and exchange information through the network cloud 215. In some examples, one or more of: the user device 205, the demand/supply matching system 210, the practice management system 220, or the practice management server 225 may include various applications implemented as processor-executable program instructions. In some embodiments, various processor-executable program instruction applications may also be used to manipulate information stored remotely and process and analyze data stored remotely across the network cloud 215 (for example, the Internet). According to an exemplary embodiment, as shown in FIG. 2, exchange of information through the network cloud 215 or other network may occur through one or more high speed connections. In some cases, high speed connections may be over-the-air (OTA), passed through networked systems, directly connected to one or more network cloud 215 or directed through one or more router. In various implementations, one or more router may be optional, and other embodiments in accordance with the present disclosure may or may not utilize one or more router. One of ordinary skill in the art would appreciate that there are numerous ways any or all of the depicted devices may connect with the network cloud 215 for the exchange of information, and embodiments of the present disclosure are contemplated for use with any method for connecting to networks for the purpose of exchanging information. Further, while this application may refer to high speed connections, embodiments of the present disclosure may be utilized with connections of any useful speed. In an illustrative example, components or modules of the system may connect to one or more of: the user device 205, the demand/supply matching system 210, the practice management system 220, or the practice management server 225 via the network cloud 215 or other network in numerous ways. For instance, a component or module may connect to the system i) through a computing device directly connected to the network cloud 215, ii) through a computing device connected to the network cloud 215 through a routing device, or iii) through a computing device connected to a wireless access point. One of ordinary skill in the art will appreciate that there are numerous ways that a component or module may connect to a device via network cloud 215 or other network, and embodiments of the present disclosure are contemplated for use with any network connection method. In various examples, one or more of: the user device 205, the demand/supply matching system 210, the practice management system 220, or the practice management server 225 could include a personal computing device, such as a smartphone, tablet computer, wearable computing device, cloud-based computing device, virtual computing device, or desktop computing device, configured to operate as a host for other computing devices to connect to. In some examples, one or more communications means of the system may be any circuitry or other means for communicating data over one or more networks or to one or more peripheral devices attached to the system, or to a system module or component. Appropriate communications means may include, but are not limited to, wireless connections, wired connections, cellular connections, data port connections, Bluetooth® connections, near field communications (NFC) connections, or any combination thereof. One of ordinary skill in the art will appreciate that there are numerous communications means that may be utilized with embodiments of the present disclosure, and embodiments of the present disclosure are contemplated for use with any communications means.

FIG. 3 depicts a structural view of an exemplary computing device adapted with an embodiment Demand/Supply Matching Engine (DSME) configured to execute a process scheduling service provider and service consumer interactions based on determining service consumer scores, service provider scores, and service time slot scores predicting outcomes for provider service to a consumer in a time slot, and automatically adapting demand and supply matched as functions of the scores. In FIG. 3, the block diagram of the exemplary demand/supply matching system 210 includes processor 305 and memory 310. The processor 305 is in electrical communication with the memory 310. The depicted memory 310 includes program memory 315 and data memory 320. The depicted program memory 315 includes processor-executable program instructions implementing the DSME (Demand/Supply Matching Engine) 325. In some embodiments, the illustrated program memory 315 may include processor-executable program instructions configured to implement an OS (Operating System). In various embodiments, the OS may include processor executable program instructions configured to implement various operations when executed by the processor 305. In some embodiments, the OS may be omitted. In some embodiments, the illustrated program memory 315 may include processor-executable program instructions configured to implement various Application Software. In various embodiments, the Application Software may include processor executable program instructions configured to implement various operations when executed by the processor 305. In some embodiments, the Application Software may be omitted. In the depicted embodiment, the processor 305 is communicatively and operably coupled with the storage medium 330. In the depicted embodiment, the processor 305 is communicatively and operably coupled with the I/O (Input/Output) interface 335. In the depicted embodiment, the I/O interface 335 includes a network interface. In various implementations, the network interface may be a wireless network interface. In some designs, the network interface may be a Wi-Fi interface. In some embodiments, the network interface may be a Bluetooth interface. In an illustrative example, the exemplary demand/supply matching system 210 may include more than one network interface. In some designs, the network interface may be a wireline interface. In some designs, the network interface may be omitted. In the depicted embodiment, the processor 305 is communicatively and operably coupled with the user interface 340. In various implementations, the user interface 340 may be adapted to receive input from a user or send output to a user. In some embodiments, the user interface 340 may be adapted to an input-only or output-only user interface mode. In various implementations, the user interface 340 may include an imaging display. In some embodiments, the user interface 340 may include an audio interface. In some designs, the audio interface may include an audio input. In various designs, the audio interface may include an audio output. In some implementations, the user interface 340 may be touch-sensitive. In some designs, the exemplary demand/supply matching system 210 may include an accelerometer operably coupled with the processor 305. In various embodiments, the exemplary demand/supply matching system 210 may include a GPS module operably coupled with the processor 305. In an illustrative example, the exemplary demand/supply matching system 210 may include a magnetometer operably coupled with the processor 305. In some embodiments, the user interface 340 may include an input sensor array. In various implementations, the input sensor array may include one or more imaging sensor. In various designs, the input sensor array may include one or more audio transducer. In some implementations, the input sensor array may include a radio-frequency detector. In an illustrative example, the input sensor array may include an ultrasonic audio transducer. In some embodiments, the input sensor array may include image sensing subsystems or modules configurable by the processor 305 to be adapted to provide image input capability, image output capability, image sampling, spectral image analysis, correlation, autocorrelation, Fourier transforms, image buffering, image filtering operations including adjusting frequency response and attenuation characteristics of spatial domain and frequency domain filters, image recognition, pattern recognition, or anomaly detection. In various implementations, the depicted memory 310 may contain processor executable program instruction modules configurable by the processor 305 to be adapted to provide image input capability, image output capability, image sampling, spectral image analysis, correlation, autocorrelation, Fourier transforms, image buffering, image filtering operations including adjusting frequency response and attenuation characteristics of spatial domain and frequency domain filters, image recognition, pattern recognition, or anomaly detection. In some embodiments, the input sensor array may include audio sensing subsystems or modules configurable by the processor 305 to be adapted to provide audio input capability, audio output capability, audio sampling, spectral audio analysis, correlation, autocorrelation, Fourier transforms, audio buffering, audio filtering operations including adjusting frequency response and attenuation characteristics of temporal domain and frequency domain filters, audio pattern recognition, or anomaly detection. In various implementations, the depicted memory 310 may contain processor executable program instruction modules configurable by the processor 305 to be adapted to provide audio input capability, audio output capability, audio sampling, spectral audio analysis, correlation, autocorrelation, Fourier transforms, audio buffering, audio filtering operations including adjusting frequency response and attenuation characteristics of temporal domain and frequency domain filters, audio pattern recognition, or anomaly detection. In the depicted embodiment, the processor 305 is communicatively and operably coupled with the multimedia interface 345. In the illustrated embodiment, the multimedia interface 345 includes interfaces adapted to input and output of audio, video, and image data. In some embodiments, the multimedia interface 345 may include one or more still image camera or video camera. In various designs, the multimedia interface 345 may include one or more microphone. In some implementations, the multimedia interface 345 may include a wireless communication means configured to operably and communicatively couple the multimedia interface 345 with a multimedia data source or sink external to the demand/supply matching system 210. In various designs, the multimedia interface 345 may include interfaces adapted to send, receive, or process encoded audio or video. In various embodiments, the multimedia interface 345 may include one or more video, image, or audio encoder. In various designs, the multimedia interface 345 may include one or more video, image, or audio decoder. In various implementations, the multimedia interface 345 may include interfaces adapted to send, receive, or process one or more multimedia stream. In various implementations, the multimedia interface 345 may include a GPU. In some embodiments, the multimedia interface 345 may be omitted. Useful examples of the illustrated demand/supply matching system 210 include, but are not limited to, personal computers, servers, tablet PCs, smartphones, or other computing devices. In some embodiments, multiple demand/supply matching system 210 devices may be operably linked to form a computer network in a manner as to distribute and share one or more resources, such as clustered computing devices and server banks/farms. Various examples of such general-purpose multi-unit computer networks suitable for embodiments of the disclosure, their typical configuration and many standardized communication links are well known to one skilled in the art, as explained in more detail in the foregoing FIG. 2 description. In some embodiments, an exemplary demand/supply matching system 210 design may be realized in a distributed implementation. In an illustrative example, some demand/supply matching system 210 designs may be partitioned between a client device, such as, for example, a phone, and, a more powerful server system, as depicted, for example, in FIG. 2. In various designs, a demand/supply matching system 210 partition hosted on a PC or mobile device may choose to delegate some parts of computation, such as, for example, machine learning or deep learning, to a host server. In some embodiments, a client device partition may delegate computation-intensive tasks to a host server to take advantage of a more powerful processor, or to offload excess work. In an illustrative example, some devices may be configured with a mobile chip including an engine adapted to implement specialized processing, such as, for example, neural networks, machine learning, artificial intelligence, image recognition, audio processing, or digital signal processing. In some embodiments, such an engine adapted to specialized processing may have sufficient processing power to implement some features. However, in some embodiments, an exemplary demand/supply matching system 210 may be configured to operate on a device with less processing power, such as, for example, various gaming consoles, which may not have sufficient processor power, or a suitable CPU architecture, to adequately support demand/supply matching system 210. Various embodiment designs configured to operate on a such a device with reduced processor power may work in conjunction with a more powerful server system.

FIG. 4 depicts an illustrative process flow of an exemplary Demand/Supply Matching Engine (DSME) automatically adapting demand and supply matched as functions of service consumer scores, service provider scores, and service time slot scores, in accordance with some embodiment implementations. The method depicted in FIG. 4 is given from the perspective of the DSME 325 implemented via processor-executable program instructions executing on the demand/supply matching system 210 processor 305, depicted in FIG. 3. In the illustrated embodiment, the DSME 325 executes as program instructions on the processor 305 configured in the DSME 325 host demand/supply matching system 210, depicted in at least FIG. 2 and FIG. 3. In some embodiments, the DSME 325 may execute as a cloud service communicatively and operatively coupled with system services, hardware resources, or software elements local to and/or external to the DSME 325 host demand/supply matching system 210. The depicted method 400 begins at step 405 with the processor 305 creating an N×M matrix representing N providers and M time slots. Then, the method continues at step 410 with the processor 305 computing a probability score for each entry (Nᵢ, Mⱼ) for no-shows, delays, or cancellation. In various embodiment designs, the method may repeat.

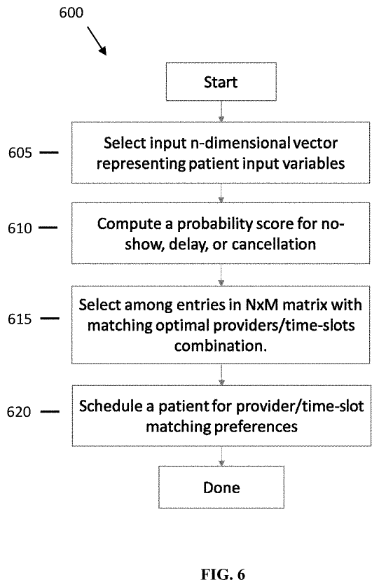

FIG. 5 depicts an illustrative process flow of an exemplary Demand/Supply Matching Engine (DSME) automatically adapting demand and supply matched as functions of service consumer scores, service provider scores, and service time slot scores, in accordance with some embodiment implementations. The method depicted in FIG. 5 is given from the perspective of the DSME 325 implemented via processor-executable program instructions executing on the demand/supply matching system 210 processor 305, depicted in FIG. 3. In the illustrated embodiment, the DSME 325 executes as program instructions on the processor 305 configured in the DSME 325 host demand/supply matching system 210, depicted in at least FIG. 2 and FIG. 3. In some embodiments, the DSME 325 may execute as a cloud service communicatively and operatively coupled with system services, hardware resources, or software elements local to and/or external to the DSME 325 host demand/supply matching system 210. The depicted method 500 begins at step 505 with the processor 305 selecting an N×M matrix consisting of probability scores for no-show, delay, or cancellation. Then, the method continues at step 510 with the processor 305 assigning an occupancy state representing whether each provider/time slot combination in the selected N×M matrix is filled or not. In some embodiment implementations, the method may repeat.

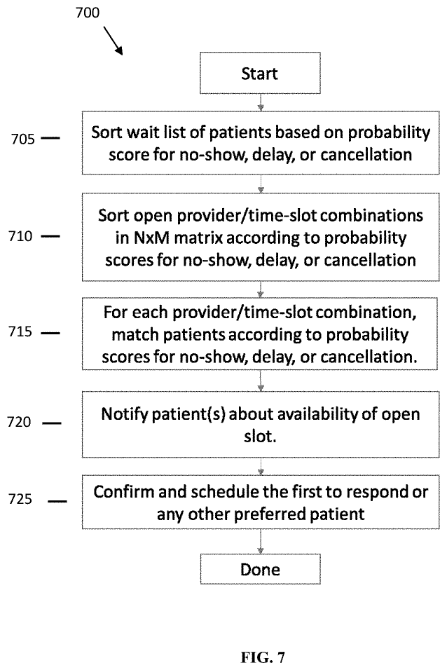

FIG. 6 depicts an illustrative process flow of an exemplary Demand/Supply Matching Engine (DSME) automatically adapting demand and supply matched as functions of service consumer scores, service provider scores, and service time slot scores, in accordance with some embodiment implementations. The method depicted in FIG. 6 is given from the perspective of the DSME 325 implemented via processor-executable program instructions executing on the demand/supply matching system 210 processor 305, depicted in FIG. 3. In the illustrated embodiment, the DSME 325 executes as program instructions on the processor 305 configured in the DSME 325 host demand/supply matching system 210, depicted in at least FIG. 2 and FIG. 3. In some embodiments, the DSME 325 may execute as a cloud service communicatively and operatively coupled with system services, hardware resources, or software elements local to and/or external to the DSME 325 host demand/supply matching system 210. The depicted method 600 begins at step 605 with the processor 305 selecting an input n-dimensional vector representing patient input variables. Then, the method continues at step 610 with the processor 305 computing a probability score for no-show, delay, or cancellation by the patient. Then, the method continues at step 615 with the processor 305 selecting among entries in an N×M matrix with matching optimal providers/time slots combination. Then, the method continues at step 620 with the processor 305 scheduling a patient for provider/time slot matching preferences. In various embodiments, the method may repeat.

FIG. 7 depicts an illustrative process flow of an exemplary Demand/Supply Matching Engine (DSME) automatically adapting demand and supply matched as functions of service consumer scores, service provider scores, and service time slot scores, in accordance with some embodiment implementations. The method depicted in FIG. 7 is given from the perspective of the DSME 325 implemented via processor-executable program instructions executing on the demand/supply matching system 210 processor 305, depicted in FIG. 3. In the illustrated embodiment, the DSME 325 executes as program instructions on the processor 305 configured in the DSME 325 host demand/supply matching system 210, depicted in at least FIG. 2 and FIG. 3. In some embodiments, the DSME 325 may execute as a cloud service communicatively and operatively coupled with system services, hardware resources, or software elements local to and/or external to the DSME 325 host demand/supply matching system 210. The depicted method 700 begins at step 705 with the processor 305 sorting a wait list of patients based on the probability score for no-show, delay, or cancellation. Then, the method continues at step 710 with the processor 305 sorting open provider/time slot combinations in an N×M matrix according to probability scores for no-show, delay, or cancellation. Then, the method continues at step 715 with the processor 305 performing a loop operation, wherein for each provider/time slot combination, the processor 305 matches patients to provider/time slot combinations according to probability scores for no-show, delay, or cancellation. Then, the method continues at step 720, with the processor 305 notifying patients about availability of an open provider/time slot combination. Then, the method continues at step 725, with the processor 305 confirming and scheduling the first to respond, or any other preferred patient. In some embodiments, the method may repeat.

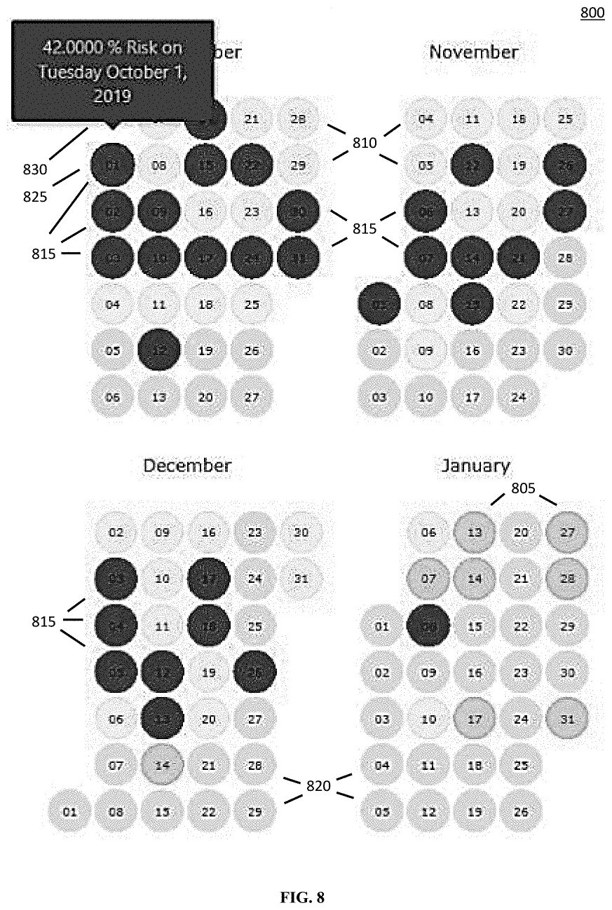

FIG. 8 depicts an illustrative risk heatmap exemplary of an embodiment Demand/Supply Matching Engine (DSME) automatically adapting demand and supply matched as functions of service consumer scores, service provider scores, and service time slot scores, in accordance with some embodiment implementations. In FIG. 8, the exemplary risk heatmap 800 highlights actionable insights for service providers. In the depicted embodiment, the color coded risk heatmap 800 depicts the percentage risk for a given day based on predicted no-shows, delays and cancellations. In some embodiments, the percentage risk may be visually presented to a user via a user interface configured in the user's computing device. In some examples, the percentage risk may be calculated, and the calculated percentage risk used by an embodiment Demand/Supply Matching Engine (DSME) to match demand and supply. In the illustrated embodiment, the highlighted percentage risk indicators are a subset of the key performance indexes (KPIs) offered, configured to highlight actionable performance insights for the user. In the depicted embodiment, the color coded risk heatmap 800 displays a portion of a monthly calendar including the percentage risk for each day in each month. In the illustrated example, the risk heatmap 800 includes risk indicators for days with low risk 805, moderate risk 810, and high risk 815. In some embodiments, the color indicating each level of risk may be determined as a function of a user-selected threshold configured for each day. In various implementations, one or more threshold governing the color indicating risk level may be determined as a function of a predictive analytic model trained as a function of historical outcomes. In the illustrated embodiment, the risk heatmap includes risk indicators for days with unknown risk 820, for days on which risk has not been calculated. In some examples, risk indicators for unknown risk may be presented for a given day when risk cannot be estimated within a predetermined confidence interval, which may be the case on a given day for various reasons that may include, for example, insufficient data for a consumer, provider, or time slot. In the depicted embodiment, the user may select a risk indicator to access additional detail concerning the risk indicated for the day associated to the risk indicator. In the illustrated embodiment, in response to the user selecting the high risk day 825 to access additional detail concerning risk on that day, the user is presented with the additional risk data 830 describing the risk for that day. In the depicted example, the additional risk data 830 is presented as a balloon popup displayed by a user interface. In various examples, the percentage risk for each day in each month may be the percentage risk on each day for a no-show, delay, or cancellation, by a consumer, patient, or client. In some embodiments, the percentage risk for each day in each month may be the percentage risk on each day for a no-show, delay, or cancellation, by a provider, doctor, or professional.

Although various embodiments have been described with reference to the Figures, other embodiments are possible. For example, various embodiments' described methods may assign scores to clients, providers, and time slots in a linked database, such as a database of scheduling status reports, clinical reports, claims data, or other such database. In some designs, historical data may be reviewed to characterize population trends and identify key predictive variables such as demographic, scheduling, administrative, clinical, or other factors that may influence the planning, organizing, tracking, evaluating, and other relevant activities involved in scheduling and work flow management. In an example illustrative of various embodiment implementations' design and usage, a score assigned to individual clients, providers, and time slots may be calculated from predictive variables of a client population within which the client belongs and the practice environment in which care is delivered. In various embodiment designs, scores may be related to probability assignments of likelihood and no-shows, delays, or cancellations. In an illustrative example, these probability estimates for clients, providers, and time slots are collectively optimized to arrive at a schedule with maximum likelihood of having a full schedule and optimizing resource utilization and revenue expenditure. An embodiment scheduling decision support system and scheduler design are described based on the methods of optimized probabilistic demand and supply matching. The exemplary methods and systems described are useful for, however not limited to, enhancing performance of scheduling in an environment where clients and practice environments have large variation. Exemplary usage of various embodiment system and method designs may advantageously minimize no-shows, delays, and cancellations, which directly affect resource allocation and utilization, practice revenue, and improve client and provider satisfaction and lowers wait times. In an illustrative example, various embodiment methods, approaches, and systems described can be generalized and applied to any service rendering environment that is dependent on scheduling events and client and service provider interactions.

Although some prior art technical practice management solutions exist, difficulties remain in translating theorized benefits into actual gains for practices. For example, in various scenarios exemplary of prior art practice management solutions' design and usage, missed appointments remain a significant source of inefficiency and lost revenue. In an illustrative example, a review of 12-year data from 10 regional hospitals' scheduling options and reminder systems including phone calls, text messaging, email notification, and the like (Kheirkhah P, Feng Q, Travis L M, Tavakoli-Tabasi S, Sharafkhaneh A. Prevalence, predictors and economic consequences of no-shows. BMC Health Sery Res. 2016 Jan. 14; 16:13. doi: 10.1186/s12913-015-1243-z. PMID: 26769153; PMCID: PMC4714455) showed no-show rates exceed on average 18%. Data indicated “that no-show imposed a major burden on this health care system. Further, implementation of a reminder system only modestly reduced the no-show rate.” Similarly, a comprehensive study with over 6600 participants (Gurol-Urganci Ii, de Jongh T, Vodopivec-Jamsek V, Atun R, Car J. Mobile phone messaging reminders for attendance at healthcare appointments. 2013 Dec. 5; (12):CD007458. doi: 10.1002/14651858.CD007458.pub3) found “the attendance to appointment rates were 67.8% for the no reminders group, 78.6% for the mobile phone messaging reminders group and 80.3% for the phone call reminders group.” These assessments show practices still face major hurdles in overcoming no show issues despite advances in practice management solutions with an average no-show rate of 23% across all specialties (Leila F. Dantas, Julia L. Fleck, Fernando L. Cyrino Oliveira, Silvio Hamacher. No-shows in appointment scheduling—a systematic literature review. Department of Industrial Engineering, Pontifical Catholic University of Rio de Janeiro, Rua Marquês de São Vicente, 225, Rio de Janeiro, R J, 22451-900, Brazil). In view of such research data, prior art best practices of auto-messaging notifications and reminders fall short of reducing the problem significantly.

In contrast with prior art practice management solutions, various embodiment implementations in accordance with the present disclosure provide improved practice management solutions. Such improved practice management may be a result of various embodiments' focus on targeted issue resolution. In an illustrative example, workflow optimization including addressing the no-show rates has significant ramification for practice performance on resource utilization, quality and outcome measures, and smoother operations. Various examples may leverage an understanding of factors contributing to no-show rates based on insights identifying and implementing targeted remedies (for example, studies have shown no-shows were associated among others with satellite clinics, new client visits, younger age, and insurance type).

In some prior art practice management solutions, no-show rates are particularly higher for subspecialties. In an illustrative example, various embodiment practice management implementations reduce or eliminate this issue, based on real-time, probabilistic demand/supply matching of available slots with interested clients. In various embodiment designs providing a solution to this two-sided problem, the supply-side (available provider slots) may be fixed and the demand-side may be variable. For example, to maximize effective matching, a practice would need to keep a candidate list/wish list of clients with probabilistic estimates. Such a probabilistic estimate may contain weighting factors such as lead time, convenience, time flexibility, distance, transportation options, age, insurance coverage, previous no-show history, demographic and socio-economic factors, and the like. Such a system may best be served by a GPS functionality, which clients can opt into, to track client's current location and movement towards the practice. In some embodiments, this capability may be offered as part of an integrated and connected health solution, which may be used for providing clients a platform for all touch points pre, during, and post care delivery. Some embodiment implementations may include a check-in process configured to assist an embodiment Probabilistic Demand/Supply Matching Algorithm to be more effective in reducing no-shows, delays, and cancellations. In an example illustrative of various check-in process embodiments' design and usage, some designs may adjust the scores of a consumer or provider if the consumer or provider checks-in a short time before an appointment, to confirm the appointment. Some check-in process embodiments may adjust the scores of a consumer or provider if the consumer or provider does not check-in within a predetermined check-in time. In various embodiments, consumer or provider scores adjusted in response to the check-in behavior of the consumer or provider may be used to determine wait list priority for a consumer or work assignment for aa provider. Other factors that can also help improve performance may include: Nudging, implementing ideas from behavioral science to entice and incentivize clients to respond to notifications (rewards, surprises, coupons, discounted advance pay option, and the like); and, Limiting/eliminating infrastructural barriers that prevent clients from accepting the open slot (for example providing Uber/Lyft rides and parking passes). Such expenses may pale by comparison to the average ˜$200 practices lose per canceled appointment. Some practices often tolerate numerous ‘no shows’ from individuals, while others discharge clients after a 3rd violation, for example. Encouraging individuals aggressively to sign up for a GPS based tracker/notifier smartphone app to reduce ‘no shows’ and facilitating rescheduling, especially after the first violation, could yield significant results. ‘No shows’ have a significant negative effect on scheduling, as appointments missed must be rescheduled for the next available slot. Reducing ‘no shows’ not only helps the bottom-line of practices but increases the overall client satisfaction by reducing scheduling wait-time. For a busy practice, seeing thousands of clients every month, a successful reduction of ‘no shows’ should automatically lead to significant reductions in scheduling wait-times.

Although various practice management and workflow optimization systems do exist, a solution to the glaring ‘client no show’ issue is still lacking. In contrast with prior art practice management systems, various embodiment implementations in accordance with the present disclosure may provide an adaptive, machine-learning leveraged, real-time, probabilistic demand/supply matching method and system permitting significantly lower client ‘no shows’, improve the practice bottom-line and increase overall client and provider satisfaction. In an illustrative example, some embodiment designs may employ a data-driven approach to identify the root-cause of ‘no shows’ at a particular clinic, unlocking the possibility of further offerings such as: Integration with Parking providers around the clinic for an advance parking spot purchase at a discount; Integration with transportation providers such as Uber/Lyft and non-emergency medical transportation; Payment assistance programs for clients with financial constraints; or, Integrating and enabling clients to locate screened and ethical child care providers.

Various embodiments disclosed hereinabove may be summarized as follows:

### Embodiment 1

In an illustrative example, an embodiment implementation in accordance with the present disclosure may include an integrated decision support process, the process comprising: gathering and characterizing historical scheduling, clinical, demographic, administrative and other data about appointment status trends for a given practice; defining, storing, and tracking quantifiable performance indicators over time showing trends and patterns in no-shows, cancelations, delays, and other similar undesirable outcomes; evaluating historical data to identify key variables that have impact on outcomes under consideration (for example, no-shows, cancellations, delays, and the like) including client, provider, and practice attributes as well as broad parameters such as population level geographic and socioeconomic indicators, weather, traffic patterns, and other relevant variables, wherein examples of data points may include, but not limited to, scheduled Day, Clinic Appointment Day, Clinic Appointment Time (AM or PM), Gender, Age, Marital Status, Number of Children, Distance to clinic, New referral vs. old, Insurance coverage, Recent insurance change, Urgency of care needed, Chronic conditions, Days since the last visit, Previous no-show history, Number of prior visits, Notification/reminder sent or not, Type of notification/reminder, Confirmation received or not, Clinic appointment outcome, and the like; characterizing demand patterns, trends, and cycles for the specific population served by a given practice to build a demand forecasting model based on influential variables; modeling the interaction between variables of interest (first-order, second-order, etc.) and assigning influence scores on each interaction and the broader outcome of interest using concepts and tools including, but not limited to, regression analysis, decision trees, neural networks, clustering, linear algebra, and any other mathematical methods and analyses; aggregating variable scores to generate cumulative scores for predicting of a client's no-show probability, a provider's performance, and probability of no-show, delay, and cancellation for a given time slot; classification of outcomes (for example, show versus no-show) based on an adjustable threshold parameter, and using such classification to characterize and make predictions about client and broad population level behaviors; defining, modeling, storing, and tracking available time slots, and scoring and ranking available time slots according to practice's ascribed value relative to a desired outcome; defining, modeling, storing, and tracking providers, and scoring and ranking them according to practice's ascribed value relative to a desired outcome; defining, modeling, storing, and tracking a list of clients, and scoring and ranking them according to probability of no-show, delays, and/or cancellation; defining, modeling, storing, and tracking a list of clients, and scoring and ranking them according to scheduling preference; defining, modeling, storing, and tracking a list of clients, and scoring and ranking them according to acuity of care needed; implementing an adaptive algorithm utilizing cumulative scores for optimizing maximum possible scheduling efficiency based on the aggregate of the most optimal matching pairs to schedule a client; implementing real-time predictive, intelligent, and adaptive scheduling system based on probabilistic estimate of no-shows, delays, and cancellations, practice resource utilization/allocation preferences, demand forecasting, and other related factors; implementing real-time assessment of no-show, delay, and cancellation risk for matching pairs of client and slot (in the context of dynamically tracked internal to the practice and external variables; aggregating overall performance per desired period (hourly, daily, weekly, etc.) and classifying risk of resource under and/or over-utilizations; adaptive and automated cancellation and re-scheduling of clients based on matching preference and scores; adaptive and automated scheduling to fill open slots upon cancellation from a list of scored and ranked client wait list; application programming interface (API) and/or other forms of integration schemes into existing electronic health records, practice management, and other software and systems; measuring, optimizing, and tracking key performance indexes (KPIs) considering real-time data and client feedback; dynamic tracking and real-time update of variables, scores, and applicable parameters depending on past, prevailing, and/or projected conditions; display in any form factor comprising of optimal scheduling output; dashboard in any form factor comprising of presentations of KPIs in any form for the benefit of evaluating tracking performance per desired time period (hourly, daily, weekly, and the like); applications, browsers, or any other forms of user interface for gathering inputs, analyzing data, scheduling and/or rescheduling, tracking list of clients, providers, and available slots, displaying optimal schedule, displaying scheduling performance, and other functionality involved in creating a scheduled event; and, adaptive and automated communication system for interaction with clients (schedule in-take, notifications, reminders, confirmation, and the like).

### Embodiment 2

In an illustrative example, an embodiment design in accordance with the present disclosure may include a computer implemented method of scoring a plurality of clients, providers, and time slots, the method comprising: obtaining a plurality of client or clients, providers, and time slots, wherein at least some of the client or clients have previously used the practice where care is rendered by providers, and wherein at least some of the providers having rendered care services to at least some of the clients; tracking and evaluating each client-provider interaction for scheduling performance on no-shows, delays, or cancellations; identifying key factors contributing to scheduling performance; identifying a weighting factor for each of the key factors contributing to client's score and adjusting the score of each of the clients based on the weighting factors; identifying a weighting factor for each of the key factors contributing to provider's score and adjusting the score of each of the providers based on the weighting factors; identifying a weighting factor for each of the key factors contributing to time slot's score and adjusting the score of each of the time slot based on the weighting factors; identifying a weighting factor for practice's preference for providers, clients, and/or time slots and adjusting the score of each client, provider, and/or time slot based on the weighting factors; identifying a weighting factor for practice's preference for providers, clients, and/or time slots and adjusting the score of each client, provider, and/or time slot based on the weighting factors; identifying a weighting factor for client's preference for providers and/or time slots and adjusting the score of each client, provider, and/or time slot based on the weighting factors; identifying a weighting factor for internal and/or external environmental and other constraints that simultaneously or in combination influence providers, clients, and/or time slots performance and adjusting the score of each of client, provider, and/or time slot based on the weighting factors; assigning a score to each client, provider, and time slot based on outcomes of a scheduled event; assigning a score to each client, provider, and time slot based on changes of key variables over time; and, dynamically tracking and updating in real-time scores and applicable parameters depending on past, prevailing, and/or projected conditions.

### Embodiment 3

In an illustrative example, an embodiment implementation in accordance with the present disclosure may include a computer implemented process including scheduling and scheduler design for a plurality of clients, providers, and time slots, the process comprising: methods and processes of Embodiment 2 in any order or combination; gathering input from clients on key variables; if new client, creating an entry in a database consisting of key variables and associated values; for old client, searching the client from a database and checking if any of the variables have changed since last interaction; updating client score based on input variables; gathering a provider's disposition of client's request; presenting a select list of available slots based on algorithmic matching of client, provider, and time slot scores; booking and confirming a schedule; overbooking additional clients for slots with higher likelihood of no-show or cancellation; keeping an updated wait list of clients sorted by probability score for no-show, delay, or cancellation; keeping an updated list of provider/time slot availability sorted by probability score for no-show, delay, or cancellation; matching clients with available slots on events of cancellation based on probability scores for no-show, delay, or cancellation; notifying clients of availability of slot(s) and tracking responses, and scheduling first to respond or any other preferred client; providing a summary of no-show, delay, cancellation patterns, trends, and performance per time period of interest (hourly, daily, weekly, and the like); and, implementing an adaptive and intelligent scheduling using scores for clients, providers, and time slots depending on any past, prevailing, and/or projected conditions.

### Embodiment 4

In an illustrative example, an embodiment implementation in accordance with the present disclosure may include Embodiment 1 and Embodiment 2 applied for any other service rendering entity consisting of scheduled client and service provider interaction, wherein client is generalized to any client, and wherein provider is generalized to any service provider under any kind of service rendering environment.

### Embodiment 5

In an illustrative example, an embodiment implementation in accordance with the present disclosure may include process and decision support elements of Embodiment 1 and Embodiment 2 applied for any other service rendering entity consisting of scheduled client and service provider interaction, wherein client is generalized to any client, and wherein provider is generalized to any service provider under any kind of service rendering environment.

In the Summary above and in this Detailed Description, and the Claims below, and in the accompanying drawings, reference is made to particular features of various embodiments of the invention. It is to be understood that the disclosure of embodiments of the invention in this specification is to be interpreted as including all possible combinations of such particular features. For example, where a particular feature is disclosed in the context of a particular aspect or embodiment of the invention, or a particular claim, that feature can also be used—to the extent possible—in combination with and/or in the context of other particular aspects and embodiments of the invention, and in the invention generally.

While multiple embodiments are disclosed, still other embodiments of the present invention will become apparent to those skilled in the art from this detailed description. The invention is capable of myriad modifications in various obvious aspects, all without departing from the spirit and scope of the present invention. Accordingly, the drawings and descriptions are to be regarded as illustrative in nature and not restrictive.

It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments.

In the present disclosure, various features may be described as being optional, for example, through the use of the verb “may;”, or, through the use of any of the phrases: “in some embodiments,” “in some implementations,” “in some designs,” “in various embodiments,” “in various implementations,”, “in various designs,” “in an illustrative example,” or “for example;” or, through the use of parentheses. For the sake of brevity and legibility, the present disclosure does not explicitly recite each and every permutation that may be obtained by choosing from the set of optional features. However, the present disclosure is to be interpreted as explicitly disclosing all such permutations. For example, a system described as having three optional features may be embodied in seven different ways, namely with just one of the three possible features, with any two of the three possible features or with all three of the three possible features.

In various embodiments. elements described herein as coupled or connected may have an effectual relationship realizable by a direct connection or indirectly with one or more other intervening elements.

In the present disclosure, the term “any” may be understood as designating any number of the respective elements, i.e. as designating one, at least one, at least two, each or all of the respective elements. Similarly, the term “any” may be understood as designating any collection(s) of the respective elements, i.e. as designating one or more collections of the respective elements, a collection comprising one, at least one, at least two, each or all of the respective elements. The respective collections need not comprise the same number of elements.

While various embodiments of the present invention have been disclosed and described in detail herein, it will be apparent to those skilled in the art that various changes may be made to the configuration, operation and form of the invention without departing from the spirit and scope thereof. In particular, it is noted that the respective features of embodiments of the invention, even those disclosed solely in combination with other features of embodiments of the invention, may be combined in any configuration excepting those readily apparent to the person skilled in the art as nonsensical. Likewise, use of the singular and plural is solely for the sake of illustration and is not to be interpreted as limiting.

In the present disclosure, all embodiments where “comprising” is used may have as alternatives “consisting essentially of,” or “consisting of.” In the present disclosure, any method or apparatus embodiment may be devoid of one or more process steps or components. In the present disclosure, embodiments employing negative limitations are expressly disclosed and considered a part of this disclosure.

Certain terminology and derivations thereof may be used in the present disclosure for convenience in reference only and will not be limiting. For example, words such as “upward,” “downward,” “left,” and “right” would refer to directions in the drawings to which reference is made unless otherwise stated. Similarly, words such as “inward” and “outward” would refer to directions toward and away from, respectively, the geometric center of a device or area and designated parts thereof. References in the singular tense include the plural, and vice versa, unless otherwise noted.

The term “comprises” and grammatical equivalents thereof are used herein to mean that other components, ingredients, steps, among others, are optionally present. For example, an embodiment “comprising” (or “which comprises”) components A, B and C can consist of (i.e., contain only) components A, B and C, or can contain not only components A, B, and C but also contain one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose limit is the second number. For example, 25 to 100 mm means a range whose lower limit is 25 mm and upper limit is 100 mm.

Many suitable methods and corresponding materials to make each of the individual parts of embodiment apparatus are known in the art. According to an embodiment of the present invention, one or more of the parts may be formed by machining, 3D printing (also known as “additive” manufacturing), CNC machined parts (also known as “subtractive” manufacturing), and injection molding, as will be apparent to a person of ordinary skill in the art. Metals, wood, thermoplastic and thermosetting polymers, resins and elastomers as may be described herein-above may be used. Many suitable materials are known and available and can be selected and mixed depending on desired strength and flexibility, preferred manufacturing method and particular use, as will be apparent to a person of ordinary skill in the art.

Any element in a claim herein that does not explicitly state “means for” performing a specified function, or “step for” performing a specific function, is not to be interpreted as a “means” or “step” clause as specified in 35 U.S.C. § 112 (f). Specifically, any use of “step of” in the claims herein is not intended to invoke the provisions of 35 U.S.C. § 112 (f). Elements recited in means-plus-function format are intended to be construed in accordance with 35 U.S.C. § 112 (f).

Recitation in a claim of the term “first” with respect to a feature or element does not necessarily imply the existence of a second or additional such feature or element.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated. The drawings represent select invention embodiments; the drawings, and the represented embodiments, may be altered in many ways without departing from a claimed invention embodiment.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim in this or any application claiming priority to this application require more features than those expressly recited in that claim. Rather, as the following claims reflect, inventive aspects may lie in a combination of fewer than all features of any single foregoing disclosed embodiment. Thus, the claims following this Detailed Description are hereby expressly incorporated into this Detailed Description, with each claim standing on its own as a separate embodiment. This disclosure is to be interpreted as including all permutations of the independent claims with their dependent claims.

According to an embodiment of the present invention, the system and method may be accomplished through the use of one or more computing devices. As depicted, for example, at least in FIG. 2 and FIG. 3, one of ordinary skill in the art would appreciate that an exemplary system appropriate for use with embodiments in accordance with the present application may generally include one or more of a Central processing Unit (CPU), Random Access Memory (RAM), a storage medium (e.g., hard disk drive, solid state drive, flash memory, cloud storage), an operating system (OS), one or more application software, a display element, one or more communications means, or one or more input/output devices/means. Examples of computing devices usable with embodiments of the present invention include, but are not limited to, proprietary computing devices, personal computers, mobile computing devices, tablet PCs, mini-PCs, servers or any combination thereof. The term computing device may also describe two or more computing devices communicatively linked in a manner as to distribute and share one or more resources, such as clustered computing devices and server banks/farms. One of ordinary skill in the art would understand that any number of computing devices could be used, and embodiments of the present invention are contemplated for use with any computing device.

In various embodiments, communications means, data store(s), processor(s), or memory may interact with other components on the computing device, in order to effect the provisioning and display of various functionalities associated with the system and method detailed herein. One of ordinary skill in the art would appreciate that there are numerous configurations that could be utilized with embodiments of the present invention, and embodiments of the present invention are contemplated for use with any appropriate configuration.

According to an embodiment of the present invention, the communications means of the system may be, for instance, any means for communicating data over one or more networks or to one or more peripheral devices attached to the system. Appropriate communications means may include, but are not limited to, circuitry and control systems for providing wireless connections, wired connections, cellular connections, data port connections, Bluetooth connections, or any combination thereof. One of ordinary skill in the art would appreciate that there are numerous communications means that may be utilized with embodiments of the present invention, and embodiments of the present invention are contemplated for use with any communications means.

Throughout this disclosure and elsewhere, block diagrams and flowchart illustrations depict methods, apparatuses (i.e., systems), and computer program products. Each element of the block diagrams and flowchart illustrations, as well as each respective combination of elements in the block diagrams and flowchart illustrations, illustrates a function of the methods, apparatuses, and computer program products. Any and all such functions (“depicted functions”) can be implemented by computer program instructions; by special-purpose, hardware-based computer systems; by combinations of special purpose hardware and computer instructions; by combinations of general purpose hardware and computer instructions; and so on—any and all of which may be generally referred to herein as a “circuit,” “module,” or “system.”

While the foregoing drawings and description may set forth functional aspects of the disclosed systems, no particular arrangement of software for implementing these functional aspects should be inferred from these descriptions unless explicitly stated or otherwise clear from the context.

Each element in flowchart illustrations may depict a step, or group of steps, of a computer-implemented method. Further, each step may contain one or more sub-steps. For the purpose of illustration, these steps (as well as any and all other steps identified and described above) are presented in order. It will be understood that an embodiment can contain an alternate order of the steps adapted to a particular application of a technique disclosed herein. All such variations and modifications are intended to fall within the scope of this disclosure. The depiction and description of steps in any particular order is not intended to exclude embodiments having the steps in a different order, unless required by a particular application, explicitly stated, or otherwise clear from the context.

Traditionally, a computer program consists of a sequence of computational instructions or program instructions. It will be appreciated that a programmable apparatus (i.e., computing device) can receive such a computer program and, by processing the computational instructions thereof, produce a further technical effect.

A programmable apparatus may include one or more microprocessors, microcontrollers, embedded microcontrollers, programmable digital signal processors, programmable devices, programmable gate arrays, programmable array logic, memory devices, application specific integrated circuits, or the like, which can be suitably employed or configured to process computer program instructions, execute computer logic, store computer data, and so on. Throughout this disclosure and elsewhere a computer can include any and all suitable combinations of at least one general purpose computer, special-purpose computer, programmable data processing apparatus, processor, processor architecture, and so on.

It will be understood that a computer can include a computer-readable storage medium and that this medium may be internal or external, removable and replaceable, or fixed. It will also be understood that a computer can include a Basic Input/Output System (BIOS), firmware, an operating system, a database, or the like that can include, interface with, or support the software and hardware described herein.

Embodiments of the system as described herein are not limited to applications involving conventional computer programs or programmable apparatuses that run them. It is contemplated, for example, that embodiments of the invention as claimed herein could include an optical computer, quantum computer, analog computer, or the like.

Regardless of the type of computer program or computer involved, a computer program can be loaded onto a computer to produce a particular machine that can perform any and all of the depicted functions. This particular machine provides a means for carrying out any and all of the depicted functions.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

Computer program instructions can be stored in a computer-readable memory capable of directing a computer or other programmable data processing apparatus to function in a particular manner. The instructions stored in the computer-readable memory constitute an article of manufacture including computer-readable instructions for implementing any and all of the depicted functions.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

The elements depicted in flowchart illustrations and block diagrams throughout the figures imply logical boundaries between the elements. However, according to software or hardware engineering practices, the depicted elements and the functions thereof may be implemented as parts of a monolithic software structure, as standalone software modules, or as modules that employ external routines, code, services, and so forth, or any combination of these. All such implementations are within the scope of the present disclosure.

Unless explicitly stated or otherwise clear from the context, the verbs “execute” and “process” are used interchangeably to indicate execute, process, interpret, compile, assemble, link, load, any and all combinations of the foregoing, or the like. Therefore, embodiments that execute or process computer program instructions, computer-executable code, or the like can suitably act upon the instructions or code in any and all of the ways just described.

The functions and operations presented herein are not inherently related to any particular computer or other apparatus. Various general-purpose systems may also be used with programs in accordance with the teachings herein, or it may prove convenient to construct more specialized apparatus to perform the required method steps. The required structure for a variety of these systems will be apparent to those of skill in the art, along with equivalent variations. In addition, embodiments of the invention are not described with reference to any particular programming language. It is appreciated that a variety of programming languages may be used to implement the present teachings as described herein, and any references to specific languages are provided for disclosure of enablement and best mode of embodiments of the invention. Embodiments of the invention are well suited to a wide variety of computer network systems over numerous topologies. Within this field, the configuration and management of large networks include storage devices and computers that are communicatively coupled to dissimilar computers and storage devices over a network, such as the Internet.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

What is claimed is:

1. A computer-implemented process to match demand and supply, the process comprising:
determining service consumer scores, service provider scores, and service time slot scores predicting outcomes for provider service to a consumer in a time slot; and,
automatically adapting demand and supply matched as functions of the scores.

2. The process of claim 1, wherein determining service consumer scores, service provider scores, and service time slot scores further comprises the service consumer scores, service provider scores, and service time slot scores determined as a function of a predictive variable associated with a service consumer population within which the service consumer belongs.

3. The process of claim 1, wherein determining service consumer scores, service provider scores, and service time slot scores further comprises the service consumer scores, service provider scores, and service time slot scores determined as a function of a predictive variable associated with the service provider practice environment.

4. The process of claim 1, wherein determining service consumer scores and service provider scores further comprises the service consumer scores and service provider scores determined as a function of a probability estimate for a no-show.

5. The process of claim 1, wherein determining service consumer scores and service provider scores further comprises the service consumer scores and service provider scores determined as a function of a probability estimate for a delay.

6. The process of claim 1, wherein determining service consumer scores and service provider scores further comprises the service consumer scores and service provider scores determined as a function of a probability estimate for a cancellation.

7. The process of claim 1, wherein the process further comprises constructing a schedule with maximum likelihood of having a full schedule, based on collectively optimizing the service consumer scores, service provider scores, and service time slot scores.

8. The process of claim 1, wherein the process further comprises constructing a schedule with optimal resource utilization, based on collectively optimizing the service consumer scores, service provider scores, and service time slot scores.

9. The process of claim 1, wherein the process further comprises constructing a schedule with optimal revenue expenditure, based on collectively optimizing the service consumer scores, service provider scores, and service time slot scores.

10. The process of claim 1, wherein the process further comprises constructing a schedule with optimal wait time, based on collectively optimizing the service consumer scores, service provider scores, and service time slot scores.

11. The process of claim 1, wherein the process further comprises constructing a schedule with optimal service provider satisfaction, based on collectively optimizing the service consumer scores, service provider scores, and service time slot scores.

12. The process of claim 1, wherein the process further comprises constructing a schedule with optimal service consumer satisfaction, based on collectively optimizing the service consumer scores, service provider scores, and service time slot scores.

13. The process of claim 1, wherein the process further comprises constructing a schedule with optimal over-booking, based on collectively optimizing the service consumer scores, service provider scores, and service time slot scores.

14. The process of claim 1, wherein the process further comprises constructing a schedule with optimal under-booking, based on collectively optimizing the service consumer scores, service provider scores, and service time slot scores.

15. A computer-implemented process to match demand and supply in a service provider practice, the process comprising:
determining service consumer scores, service provider scores, and service time slot scores predicting outcomes for provider service to a consumer in a time slot, wherein the service consumer scores, service provider scores, and service time slot scores are determined as a function of a probability estimate for successful service outcome when the service consumer, service provider, and service time slot interact to satisfy the service consumer demand during the service time slot;
automatically adapting demand and supply matched as functions of the service consumer scores, service provider scores, and service time slot scores; and,
constructing a schedule with maximum likelihood of having a full schedule, based on collectively optimizing the service consumer scores, service provider scores, and service time slot scores.

16. The process of claim 15, wherein the probability estimate for successful service outcome is determined as a function of a historical service interaction involving at least two of: a service consumer, a service provider, and a service time slot.

17. The process of claim 15, wherein the service consumer score is determined as a function of at least one weighting factor for at least one key factor contributing to the service consumer score.

18. The process of claim 15, wherein the service provider score is determined as a function of at least one weighting factor for at least key factor contributing to the service provider score.

19. The process of claim 15, wherein the service time slot score is determined as a function of at least one weighting factor for at least one key factor contributing to the service time slot score.

20. The process of claim 15, wherein adapting demand and supply matched as functions of the scores further comprises identifying at least one weighting factor representing the preference of the practice for a service provider, service consumer, or service time slot characteristic or the preference of the service consumer for a service provider or service time, and adjusting the score of each service consumer, service provider, and service time slot based on the at least one weighting factor.

21. The process of claim 15, wherein collectively optimizing the service consumer scores, service provider scores, and service time slot scores further comprises assigning a score to each service consumer, service provider, and service time slot based on the historical outcome of a scheduled interaction.

22. The process of claim 15, wherein collectively optimizing the service consumer scores, service provider scores, and service time slot scores further comprises assigning a score to each service consumer, service provider, and service time slot based on planned or unplanned changes to any underlying factors affecting service consumer scores, service provider scores, and service time slot scores.

23. A computer-implemented process to match demand and supply in a service provider practice, the process comprising:
determining service consumer scores, service provider scores, and service time slot scores predicting outcomes for provider service to a consumer in a time slot, wherein the service consumer scores, service provider scores, and service time slot scores are determined as a function of a probability estimate for successful service outcome when the service consumer, service provider, and service time slot interact to satisfy the service consumer demand during the service time slot;
automatically adapting demand and supply matched as functions of the service consumer scores, service provider scores, and service time slot scores; and,
constructing a waitlist based on collectively optimizing and ranking the service consumer scores.

24. The process of claim 23, wherein the service consumer score is determined as a function of at least one weighting factor for at least one key factor contributing to the service consumer score.

25. The process of claim 23, wherein adapting demand and supply matched as functions of the scores further comprises identifying at least one weighting factor representing the preference of the practice for a service provider, service consumer, and service time slot characteristic, and adjusting the score of each service consumer based on the at least one weighting factor.

26. The process of claim 23, wherein collectively optimizing the service consumer scores, service provider scores, and service time slot scores further comprises assigning a score to each service consumer based on the historical outcome of a scheduled interaction.

27. The process of claim 23, wherein collectively optimizing the service consumer scores, service provider scores, and service time slot scores further comprises assigning a score to each service consumer based on planned or unplanned changes to any underlying factors affecting service consumer scores.

28. A computer-implemented process to match demand and supply in a service provider practice, the process comprising:
determining service consumer scores, service provider scores, and service time slot scores predicting outcomes for provider service to a consumer in a time slot, wherein the service consumer scores, service provider scores, and service time slot scores are determined as a function of a probability estimate for successful service outcome when the service consumer, service provider, and service time slot interact to satisfy the service consumer demand during the service time slot;
automatically adapting demand and supply matched as functions of the service consumer scores, service provider scores, and service time slot scores, comprising:
identifying at least one weighting factor representing the preference of the practice for a service provider, service consumer, or service time slot characteristic or the preference of the service consumer for a service provider or service time;
adjusting the score of each service consumer, service provider, and service time slot based on the at least one weighting factor;
matching a service consumer with a service provider in an available service time slot, based on the service consumer scores, service provider scores, and service time slot scores; and,
notifying the matched service consumer of the service time slot availability; and,
automatically constructing a schedule with maximum likelihood of having a full schedule, based on collectively optimizing the service consumer scores, service provider scores, and service time slot scores, comprising assigning a score to each service consumer, service provider, and service time slot based on the historical outcome of a scheduled interaction.

29. The process of claim 28, wherein a service consumer further comprises a medical patient.

30. The process of claim 28, wherein a service provider further comprises a scheduled medical service provider.

31. The process of claim 30, wherein the scheduled medical service provider further comprises a medical doctor.

32. The process of claim 28, wherein the service provider practice is a medical practice, and wherein a service time slot further comprises a period of time when the service provider practice may provide service to satisfy the service consumer demand.

33. A computer-implemented process to match demand and supply in a service provider practice, the process comprising:
determining service consumer scores, service provider scores, and service time slot scores predicting outcomes for provider service to a consumer in a time slot, wherein the service consumer scores, service provider scores, and service time slot scores are determined as a function of a probability estimate for successful service outcome when the service consumer, service provider, and service time slot interact to satisfy the service consumer demand during the service time slot;
automatically adapting demand and supply matched as functions of the service consumer scores, service provider scores, and service time slot scores, comprising:
identifying at least one weighting factor representing the preference of the practice for a service provider, service consumer, or service time slot characteristic or the preference of the service consumer for a service provider or service time;
adjusting the score of each service consumer, service provider, and service time slot based on the at least one weighting factor;
matching a service consumer with a service provider in an available service time slot, based on the service consumer scores, service provider scores, and service time slot scores; and,
notifying the matched service consumer of the service time; and,
automatically constructing a schedule with maximum likelihood of having a full schedule, based on scheduling as a function of an optimized and ranked waitlist and collectively optimizing the service consumer scores, service provider scores, and service time slot scores, wherein assigning a score to each service consumer, service provider, and service time slot is based on the historical outcome of a scheduled interaction.

34. The process of claim 33, wherein a service consumer further comprises a medical patient.

35. The process of claim 33, wherein a service provider further comprises a scheduled medical service provider.

36. The process of claim 35, wherein the scheduled medical service provider further comprises a medical doctor.

37. The process of claim 33, wherein the service provider practice is a medical practice, and wherein a service time slot further comprises a period of time when the service provider practice may provide service to satisfy the service consumer demand.

38. The process of claim 33, wherein identifying at least one weighting factor further comprises a check-in process configured to reduce no-shows, delays, or cancellations based on adapting the at least one weighting factor in response to a check-in.

39. The process of claim 33, wherein the process further comprises presenting to a user a subset of KPI offered in a color coded heatmap view highlighting actionable insights for service providers, wherein the color coded heatmap depicts the percentage risk for a given day based on predicted no-shows, delays and cancellations.
